(19) United States
(12) Patent Application Publication
(10) Pub. No.: US 2023/0028735 A1
(43) Pub. Date: Jan. 26, 2023

(54) IMPLANT DELIVERY INSTRUMENT

(71) Applicant: Pacesetter, Inc., Sylmar, CA (US)

(72) Inventors: Zachary Hanze, Porter Ranch, CA (US); Bei Ning Zhang, Pasadena, CA (US); Arees Garabed, North Hills, CA (US); Rodney Hawkins, Santa Clarita, CA (US); Xiangqun Chen, Santa Clarita, CA (US); Alexander Davis Robertson, Los Angeles, CA (US); Alex Soriano, Ventura, CA (US); Kyungmoo Ryu, Palmdale, CA (US)

(21) Appl. No.: 17/664,180

(22) Filed: May 19, 2022

Related U.S. Application Data
(60) Provisional application No. 63/223,652, filed on Jul. 20, 2021.

(51) Int. Cl. A61B 17/34; A61M 37/00

(52) U.S. Cl. CPC A61B 17/3468; A61M 37/0069; A61B 2560/063; A61B 2017/3456

(57) ABSTRACT

An implant delivery instrument includes a housing with a main body section that defines a passage therethrough from a front end of the main body section to a back end of the main body section. The passage receives an obturator through an opening at the back end, and also receives an implant device. Movement of the obturator through the passage pushes the implant device through a discharge opening at the front end and through an incision into a subdermal pocket of a patient. The housing includes a tab connected to and projecting from the front end of the main body section. The tab has a blunt dissection tip for maintaining the incision in an open state without creating or enlarging the incision, and the tab is configured to surround the implant device moving through the discharge opening along only one side of the implant device.

### CROSS-REFERENCE TO RELATED APPLICATIONS

This application claims priority to U.S. Provisional Patent Application No. 63/223,652, filed Jul. 20, 2021 and titled Implant Delivery Instrument. The subject matter of the provisional application is expressly incorporated herein by reference in its entirety.

### BACKGROUND

Embodiments of the present disclosure generally relate to the implantation of medical monitoring devices within patients, including implantable cardiac monitors (ICMs).

Subdermal implantable devices, such as ICMs, are devices that may be implanted under a patient's skin to continuously monitor the patient's cardiac activity. ICMs allow clinicians to monitor the patient's cardiac activity for an extended period of time, such as multiple years. The information recorded by ICM enables clinicians to determine if a patient complaining of symptoms has irregularities in their heart rhythm that cannot be confirmed in the clinic, particularly for transient and/or infrequent arrhythmias. For example, an ICM may be programmed to detect and record cardiac information and episodes such as atrial/ventricular tachycardia, atrial fibrillation, bradycardia, asystole, etc. The information can also aid the clinician in determining the best course of treatment for the patient. The ICM may be implanted in the patient's chest area near the sternum.

The implantation of monitoring devices, such as ICMs, is typically performed under local anesthetic using an implant tool kit, which includes an incision tool and a delivery tool. FIG. 1 illustrates an implant tool kit 10 during an implantation procedure. As shown in FIG. 1, the incision tool 12 of the tool kit 10 is used to form an incision in the skin. Then, the incision tool 12 is swapped for the delivery tool 14, and the delivery tool 14 is inserted into the incision to dissect the skin from the fat layers creating a “pocket” between the skin and the fat layers. The delivery tool 14 is then used to inject the implant device 16 (e.g., ICM) from the delivery tool 14 through the incision and into the pre-formed pocket.

A small incision is desired for patient comfort and ease of wound closure and healing post-implant. However, small incisions pose challenges for the implantation process. Using at least some known insertion systems, the incision may be difficult to keep open during the procedure to allow the implant device to pass from the delivery tool into the tissue pocket. Further, it may be difficult to maneuver the delivery tool into the tissue to correctly position the implant device at a required depth and position. For patients with taut tissue, additional force may be required to insert the implant device, while for patients with loose tissue, the implant device may move (e.g., migrate) after implant. Movement of the implant device within the pocket is undesirable as the change in position can diminish device functionality. For example, due to movement in the pocket, an ICM may be less capable of detecting cardiac signals and determining cardiac parameters, such as R-R intervals utilized in monitoring cardiac activity.

Moreover, in at least some known insertion systems, it may be difficult to push the implant device into the tissue, and this issue is exacerbated for larger implant devices. The increased force application may cause patient discomfort and delay healing. Finally, once the device is implanted, the physician may have difficulty closing the incision if the incision is damaged by the delivery tool.

A need remains for an implant delivery instrument that can safely, effectively, and accurately implant a device into a subdermal pocket in a patient for accurate patient monitoring.

### SUMMARY

In accordance with an embodiment, an implant delivery instrument is provided that includes a housing with a main body section that defines a passage therethrough from a front end of the main body section to a back end of the main body section. The passage is configured to receive an obturator through an opening at the back end, and is also configured to receive an implant device. Movement of the obturator through the passage is configured to push the implant device through a discharge opening at the front end and through an incision into a subdermal pocket of a patient. The housing includes a tab connected to and projecting from the front end of the main body section. The tab has a blunt dissection tip for maintaining the incision in an open state without creating or enlarging the incision, and the tab is configured to surround the implant device moving through the discharge opening along only one side of the implant device.

Optionally, a thickness of the tab tapers along a length of the tab from a proximal end of the tab connected to the main body section to a distal end of the tab. Optionally, a width of the tab, along at least 90% of a length of the hub, is no greater than a width of the discharge opening. Optionally, the front end of the main body section includes a leading edge that defines a hard stop surface, and the tab is connected to the hard stop surface. The tab may be rigid and integrally connected to the hard stop surface.

Optionally, the main body section has a top side and a bottom side opposite the top side. The bottom side is configured to face towards skin of the patient as the implant device is pushed through the discharge opening into the subdermal pocket. The tab is disposed along a top edge of the discharge opening between the discharge opening and the top side of the main body section. The main body section of the housing may include at least one device support wall proximate to the front end. The at least one device support wall defines a ceiling of the passage to engage the implant device and inhibit tilting of the implant device in a direction away from the tab as the obturator pushes the implant device through the discharge opening.

Optionally, a lower surface of the tab is one of angled or curved so that a distal end of the tab is closer to a center of the passage than a proximal end of the tab connected to the main body section. The shaft of the obturator may define an elongated groove along an upper surface of the shaft. The lower surface of the tab may be configured to enter the groove. Optionally, the tab includes a narrow rib that projects from a base of the tab and defines the lower surface of the tab. The narrow rib enters the groove of the shaft and the base of the tab extends above the upper surface of the shaft without entering the groove. Optionally, the narrow rib and the groove are interlocking to prevent separation of the tab and the shaft.

Optionally, the main body section of the housing defines a receptacle cavity in communication with the passage. The receptacle cavity is exposed to an external environment through a cavity opening along a side of the main body section. The receptacle cavity is configured to receive the implant device through the cavity opening.

In an embodiment, an implant delivery instrument is provided that includes an obturator and a housing. The obturator includes an elongated shaft. The housing has a main body section that has a top side and a bottom side opposite the top side. The main body section defines a passage therethrough from a front end of the main body section to a back end of the main body section. The passage is configured to receive the obturator through an opening at the back end. The passage is also configured to receive an implant device. Movement of the obturator through the passage is configured to push the implant device through a discharge opening at the front end and through an incision into a subdermal pocket of the patient. The bottom side of the main body section is configured to face towards skin of the patient as the implant device is pushed into the subdermal pocket. The housing includes a tab connected to and projecting from the front end of the main body section. The tab connects to the front end at a location between the discharge opening and the top side of the main body section.

Optionally, the tab has a blunt dissection tip for maintaining the incision in an open state without creating or enlarging the incision. Optionally, the tab is configured to surround the implant device moving through the discharge opening along only one side of the implant device. Optionally, a width of the tab, along at least 90% of a length of the hub, is no greater than a width of the discharge opening.

Optionally, the main body section of the housing includes at least one device support wall proximate to the front end. The at least one device support wall defines a ceiling of the passage to engage the implant device and inhibit tilting of the implant device in a direction away from the tab as the obturator pushes the implant device through the discharge opening.

In an embodiment, an implant delivery instrument is provided that includes an obturator and a housing. The obturator includes a shaft that defines an elongated groove along a surface of the shaft. The housing has a main body section that defines a passage therethrough from a front end of the main body section to a back end of the main body section. The passage is configured to receive the obturator through an opening at the back end. The passage is also configured to receive an implant device. Movement of the obturator through the passage is configured to push the implant device through a discharge opening at the front end and through an incision into a subdermal pocket of a patient. The housing includes a tab connected to the front end of the main body section at a location between the discharge opening and a first side of the main body section. The tab projects from the front end and is one of angled or curved so that a portion of the tab is received in the groove of the shaft of the obturator along a section of the shaft that projects out of the discharge opening.

Optionally, the tab has a blunt dissection tip for maintaining the incision in an open state without creating or enlarging the incision. The tab is configured to surround the implant device moving through the discharge opening along only one side of the implant device. Optionally, a thickness of the tab tapers along a length of the tab from a proximal end of the tab connected to the main body section to a distal end of the tab. The distal end of the tab is received in the groove to provide a smooth transition from the obturator to the tab.

### BRIEF DESCRIPTION OF THE DRAWINGS

FIG. 1 illustrates an implant tool kit during an implantation procedure.

FIG. 2 illustrates an insertion system according to an embodiment.

FIG. 3 illustrates an implant delivery instrument of the insertion system in an assembled state with an obturator of the implant delivery instrument at an advanced position relative to a housing of the implant delivery instrument.

FIG. 4 illustrates the assembled implant delivery instrument with the obturator at a retracted position relative to the housing.

FIG. 5 is a perspective view of the implant delivery instrument according to the embodiment shown in FIGS. 2 through 4.

FIG. 6 is an enlarged view of a portion of the implant delivery instrument shown in FIG. 5.

FIG. 7 is a side view of a portion of the implant delivery instrument according to the embodiment shown in FIGS. 2 through 6.

FIG. 8 illustrates a side view of the implant delivery instrument injecting the implant device into a subdermal pocket of a patient according to an embodiment.

FIG. 9 is a perspective view of portion of the implant delivery instrument according to a second embodiment.

FIG. 10 is a perspective view of the implant delivery instrument according to a third embodiment.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various method embodiments, certain operations may be omitted or added, certain operations may be combined, certain operations may be performed simultaneously and/or concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems.

The terms “subdermal” and “subcutaneous” shall mean below the skin, but not intravenous (e.g., non-transvenous). For example, an implant device within a subcutaneous pocket is not located in a chamber of the heart, in a vein on the heart, or in the lateral or posterior branches of the coronary sinus. A subdermal or subcutaneous pocket is located below the skin but above the muscle.

According to at least one embodiment, an implant delivery instrument for implanting a monitoring device into a patient is provided that has a low profile housing. The low profile housing is designed to reduce damage to the incision site tissue relative to at least some known delivery tools used to implant a monitoring device into a patient. Relative to the known delivery tools, the low profile housing also reduces the insertion force necessary to insert an end of the housing into the incision during the implantation procedure, for ensuring that the device is accurately implanted to the correct depth and position within the subdermal pocket. The housing is designed with a tab or hub that holds the upper side of the incision open while the device is injected to avoid tissue snagging on the device. The implant delivery instrument described herein is able to implant devices into small incisions with the ability to maintain incision shape before and after the implantation. One or more embodiments herein describe the implant delivery instrument being used to implant ICMs into subdermal pockets, but the implant delivery instrument can also be used to inject other types of devices into a living body.

One or more technical effects of the implant delivery instrument described herein is improved patient comfort during the implantation procedure and faster patient recovery after the procedure, relative to at least some known delivery tools. For example, the comfort and recovery can be enhanced by reducing damage to the tissue of the patient during the insertion and maneuvering of the delivery tool in the incision, and avoiding efforts to fix an incorrectly positioned device during or after the implantation procedure. Another technical effect may include requiring less force to be applied to the delivery instrument during the implantation procedure than the at least some known delivery tools, such that the delivery instrument may be easier to use and manipulate to provide more accurate device implantation than the known delivery tools.

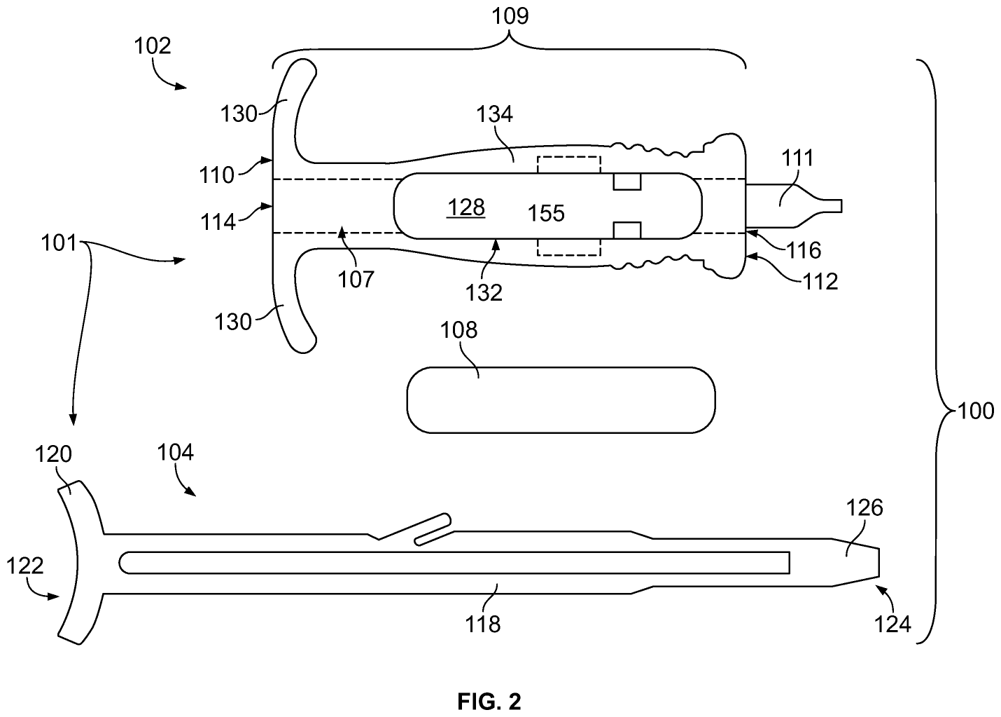

FIG. 2 illustrates an insertion system 100 according to an embodiment. The insertion system 100 includes an implant delivery instrument 101 and an implant device 108. The implant delivery instrument 101 (also referred to herein as delivery instrument and instrument) injects the implant device 108 into a subdermal pocket in the patient. The instrument 101 is shown in a disassembled state, and includes a housing 102 and an obturator 104. The obturator 104 is an introducer or plunger that is inserted within the housing 102 and is moved through the housing 102 during the implantation procedure.

The housing 102 includes a main body section 109 and a tab 111. The main body section 109 extends from first end 110 thereof to a second end 112 thereof, which is opposite the first end 110. The first end 110 is designed to be held by an entity that manipulates the instrument 101, such as a medical professional or a robotic device. The first end 110 is the end that faces away from the patient during the implantation procedure, so the first end 110 is referred to herein as a back end 110. The second end 112 faces towards the patient, and optionally may contact patient tissue, during the implantation procedure. The second end 112 is referred to herein as a front end 112. The main body section 109 defines a passage 107 therethrough. The passage 107, shown in phantom in FIG. 2, is a channel that extends the length of the main body section 109 from the front end 112 to the back end 110. The passage 107 is open along the back end 110 via a first opening 114 (referred to herein as an inlet opening), and is open along the front end 112 via a second opening 116 (referred to herein as a discharge opening).

The tab 111 is connected to the front end 112 of the main body section 109. The tab 111 projects beyond the front end 112. For example, the tab 111 extends in a forward or frontward direction from the main body section 109 based on the front/back orientation of the main body section 109. The tab 111 may extend coaxially with the passage 107 of the main body section 109. The tab 111 extends beyond the discharge opening 116.

The obturator 104 includes a shaft 118 and a handle 120. The handle 120 is at a back end 122 of the obturator 104. The shaft 118 is linearly elongated from the handle 120 to a front end 124 of the obturator 104 opposite the back end 122. The shaft 118 includes a distal tip 126 that is shaped and dimensioned to perform blunt dissection in subcutaneous tissue of the patient during the implantation procedure. For example, the distal tip 126 may be tapered, wedge-shaped, conical, or the like.

The main body section 109 may include a pair of wings 130 at the back end 110 that extend in opposite directions. The wings 130 are configured to be held between two fingers, while the user's thumb presses on the handle 120 of the obturator 104 to actuate the obturator 104 for injecting the implant device 108 into the subdermal pocket. The periphery of the main body section between the wings 130 and the front end 112 is contoured to facilitate manual gripping by the user.

The main body section 109 may define a receptacle cavity 128 in communication (e.g., fluid communication) with the passage 107. The receptacle cavity 128 is exposed to the external environment through a cavity opening 132 along a side 134 of the main body section 109. The receptacle cavity 128 is sized and shaped to receive the implant device 108 therein through the cavity opening 132. The housing 102 may include detents 155 within the receptacle cavity 128 that provide an interference fit on the implant device 108 to inhibit the implant device 108 from inadvertently falling out of the receptacle cavity 128. The receptacle cavity 128 may be located adjacent to (e.g., directly above) a portion of the passage 107, so the implant device 108 can drop from the receptacle cavity 128 into the passage 107 to inject the implant device 108. The receptacle cavity 128 has sufficient depth to hold the implant device 108 while the obturator 104 is located in the passage 107 next to (e.g., below) the implant device 108. The passage 107 includes inner dimensions that are similar to, but slightly larger than, the dimensions of the implant device 108 to enable the implant device 108 to move along the passage 107.

The implant device 108 in an embodiment is an ICM. ICMs are diagnostic tools that do not deliver pacing or shock therapies to the patient, nor do they require leads to be implanted in the patient's heart. ICMs are generally small (e.g., 1.1-1.5 cm³ in volume), and can be implanted using a small incision (e.g., 1 cm). Once inserted under the patient's skin, the ICM has a slim profile, mitigating patient concerns of comfort and aesthetics/body image. The ICM has a rectangular cross-section in the illustrated embodiment, with rounded corners and edges to avoid snagging on tissue. The ICM may have other form factors in alternative embodiments.

The ICM 108 may utilize QRS detection to determine R-R intervals utilized in connection with monitoring cardiac activity. In some ICMs, P-waves are captured from an EKG signal in order to provide evidence of sinus rhythm. An absence of P-waves is used to support a determination as to whether a patient is experiencing atrial fibrillation (AF). In subsequent analysis of the information collected by an ICM, when determining whether a patient is experiencing AF, it is desirable for clinicians to view P-wave activity in the stored data in order to facilitate diagnosis. The placement and stability of the ICM in the subdermal pocket is important for enabling the ICM to perform reliable, accurate monitoring of cardiac activity, such as for discerning the P-waves, considering that the ICM is spaced apart from the heart.

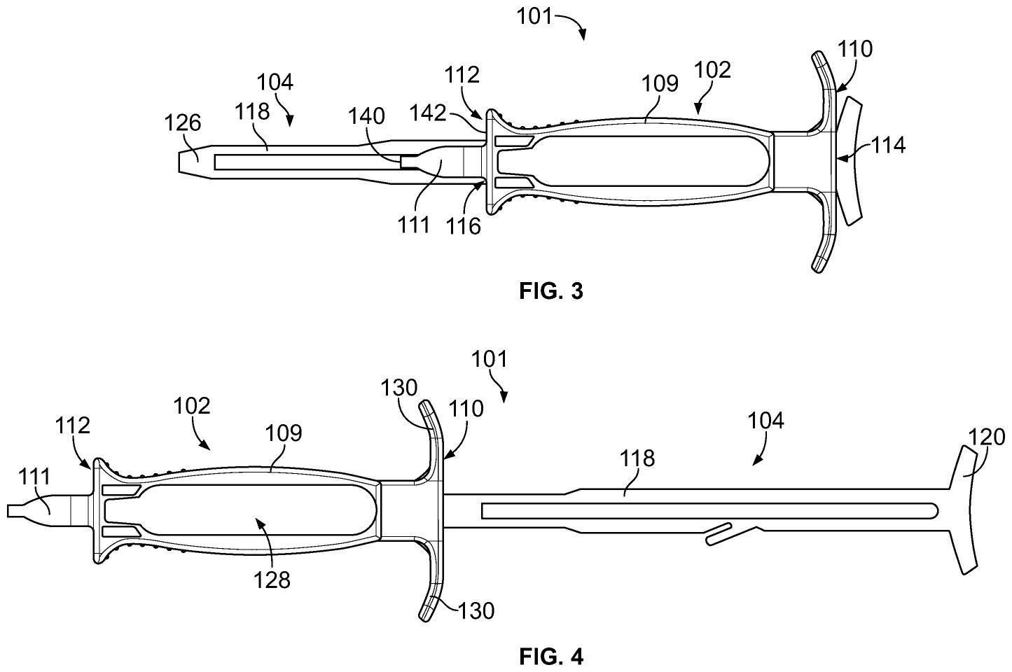

FIG. 3 illustrates the implant delivery instrument 101 in an assembled state with the obturator 104 at an advanced position relative to the housing 102. FIG. 4 illustrates the assembled implant delivery instrument 101 with the obturator 104 at a retracted position relative to the housing 102. The instrument 101 is assembled by loading the shaft 118 of the obturator 104 into the passage 107 (shown in FIG. 2) of the main body section 109 via the inlet opening 114. The implant device 108 (shown in FIG. 2) is not shown in FIG. 3 or 4.

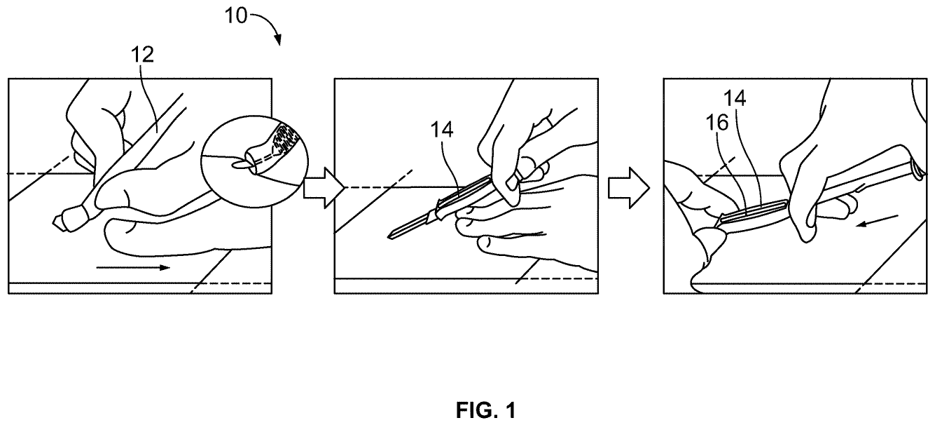

In the advanced position as shown in FIG. 3, a segment of the shaft 118 projects through the discharge opening 116 beyond the front end 112 of the main body section 109. The distal tip 126 of the obturator 104 even projects well beyond a distal end 140 of the tab 111. This exposed segment of the shaft 118 is used to perform blunt dissection in subcutaneous tissue of the patient to form a subdermal pocket. The distal tip 126 is not a cutting edge in the illustrated embodiment. The instrument 101 is utilized after an incision is made in the tissue via a discrete incision tool, as described above with reference to FIG. 1. To ensure that the pocket has a desired size and depth, the instrument 101, in the advanced position, is inserted into the incision until a leading edge 142 (e.g. surface) at the front end 112 of the main body section 109 contacts the patient tissue surrounding the incision. The leading edge 142 provides a hard stop surface to ensure accurate and repeatable pocket formation. The tab 111 may be connected to the leading edge 142. The tab 111 enters the incision with the shaft 118 of the obturator 104 such that the tab 111 is within the patient when the leading edge 142 contacts the patient tissue to provide the hard stop. This process forms the subdermal pocket.

Then, the obturator 104 is pulled back to the retracted state relative to the housing 102, as shown in FIG. 4. In the retracted state, the handle 120 is spaced apart from the wings 130, and no portion of the shaft 118 projects beyond the front end 112 of the main body section 109. The distal tip 126 (shown in FIG. 3) of the shaft 118 is proximate to the back end 110 of the main body section 109, so the obturator 104 does not block the implant device from entering the passage 107. The implant device 108 may be forced from the receptacle cavity 128 into the passage 107. Alternatively, the housing 102 may be designed to enable the implant device 108 to automatically drop into the passage upon the shaft 118 exiting the portion of the passage 107 below the receptacle cavity 128.

The instrument 101 is poised to deliver the implant device 108 into the subdermal pocket when the implant device 108 in the passage 107 and the housing 102 positioned with the tab 111 in the incision and the leading edge 142 in contact with the tissue surrounding the incision. The implant device 108 is implanted by pushing the handle 120 of the obturator 104 towards the wings 130. As the obturator 104 moves within the passage 107, the distal tip 126 pushes the implant device 108 through the discharge opening 116 and into the subdermal pocket. The instrument 101 functions similar to a syringe to inject the implant device 108 into the patient while the housing 102 is held in a desired orientation relative to the patient skin. After the implant device 108 is implanted, the user extracts the instrument 101 from the patient and closes the incision.

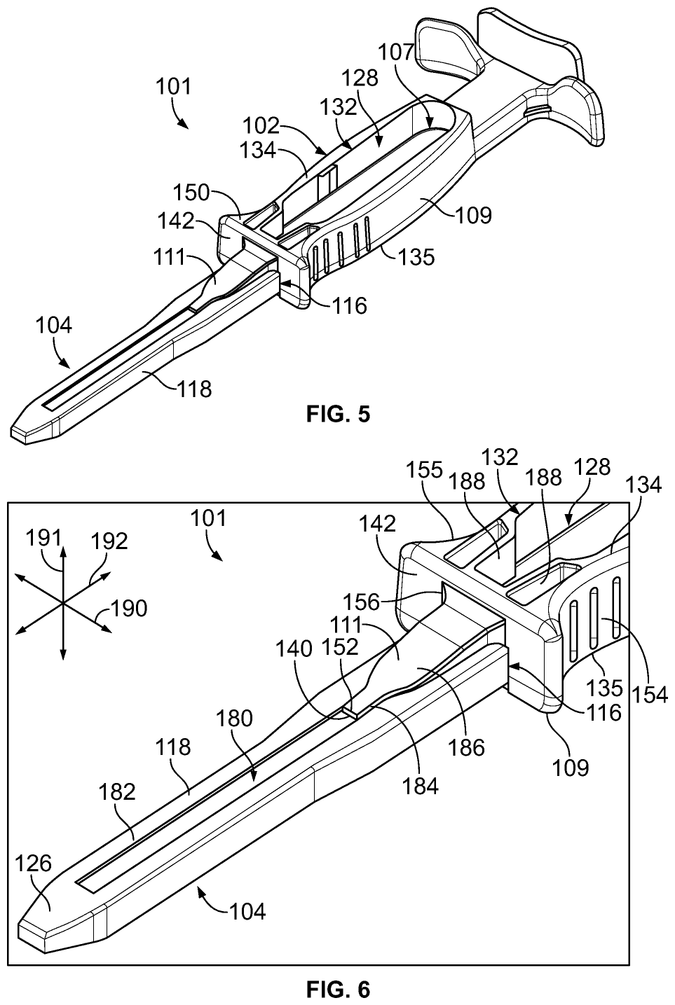

FIG. 5 is a perspective view of the implant delivery instrument 101 according to an embodiment. The instrument 101 is shown in the advanced position of the obturator 104 relative to the housing 102. The implant device 108 is not within the receptacle cavity 128, so a portion of the shaft 118 of the obturator 104 within the passage 107 is visible through the cavity opening 132. The housing 102 is a rigid material, such as plastic or metal. In an embodiment, the tab 111 is rigid and integrally connected to the leading edge 142 of the main body section 109. The tab 111 is integrally connected to the leading edge 142 at a seamless interface. For example, the tab 111 may be formed in-situ with or on the main body section 109 via additive manufacturing, molding, or the like.

In the illustrated embodiment, the leading edge 142 is a planar surface that surrounds the tab 111 and the discharge opening 116 around an entire perimeter, such as along all four sides of the tab 111 and discharge opening 116 which have rectangular cross-sections. The leading edge 142 may be disposed on a flared segment 150 of the main body section 109, such as a flange. The surface of the leading edge 142 provides a hard stop to set and limit the depth that the instrument 101 extends into the patient through the incision.

FIG. 6 is an enlarged view of a portion of the implant delivery instrument 101 shown in FIG. 5. The enlarged view shows the tab 111 and the section of the shaft 118 of the obturator 104 that projects out of the discharge opening 116 when in the advanced position.

In an embodiment, the tab 111 has a blunt dissection tip 152 at the distal end 140. The blunt dissection tip 152 is designed to maintain the incision in an open state to enable the implant device 108 (shown in FIG. 2) to be injected through the incision into the subdermal pocket without snagging on, pinching, or otherwise damaging the tissue surrounding the incision. The tab 111 does not include a cutting edge. The tab 111 does not create or enlarge the incision.

The tab 111 is designed to have a low profile to enable small incision sizes without undue stretching of the tissue at the incisions. At least one known delivery tool has a hub, rather than the tab 111, and the hub is a short tube that surrounds the entire perimeter of the obturator shaft and the implant device when extending from the housing. The tab 111 reduces the cross-sectional area of non-implanted matter that is inserted into the incision, relative to the tube-like hub. Unlike the tube-like hub, the tab 111 surrounds less than half of a perimeter of the discharge opening 116 (and less than half of the shaft 118 and implant device 108 that extend through the discharge opening 116).

In at least one embodiment, the tab 111 is a panel or wall that surrounds the discharge opening 116 along only one side thereof. It follows that the tab 111 surrounds the shaft 118 and the implant device 108, when extending through the discharge opening 116, only along one side as well. By extending only along one side, the tab 111 may reduce the cross-sectional area of the non-implanted matter inserted into the patient. To further limit the cross-sectional area, a width of the tab 111 may be no greater than a width of the discharge opening 116. The width of the discharge opening 116 extends along a width axis 190. The width axis 190 extends from a first gripping side 154 of the main body section 109 to a second gripping side 155 of the main body section 109. The discharge opening 116 has a height along a height axis 191. The shaft 118 projecting through the discharge opening 116 is oriented parallel to a longitudinal or length axis 192. The axes 190-192 are mutually perpendicular.

With additional reference to FIG. 3, the tab 111 is narrower than the portion of the shaft 118 projecting out of the discharge opening 116, so the tab 111 is narrower than the discharge opening 116. As shown in FIG. 6, a proximal end 156 of the tab 111 may broaden out and expand at the attachment location with the leading edge 142. In the illustrated embodiment, no portion of the tab 111, including the proximal end 156, is wider than the discharge opening 116. In another embodiment, only the proximal end 156 of the tab 111 is wider than the discharge opening 116. At least 90% of the length of the tab 111 from the proximal end 156 to the distal end 140 is no wider than the discharge opening 116. The narrow tab 111 avoids stretching the incision wider than the width necessary to receive the implant device 108.

In an embodiment, the tab 111 is located above the discharge opening 116 with respect to an orientation of the implant delivery instrument 101 during the implantation procedure. For example, the main body section 109 has a top side 134 and a bottom side 135 opposite the top side 134. The top side 134 may define the cavity opening 132.

FIG. 8 illustrates a side view of the implant delivery instrument 101 injecting the implant device 108 into a subdermal pocket 202 of a patient according to an embodiment. The bottom side 135 faces towards the skin 204 of the patient as the implant device 108 is pushed through the discharge opening 116 into the subdermal pocket 202. The tab 111 is connected to the leading edge 142 of the main body section 109 at a location between the discharge opening 116 and the top side 134. For example, the tab 111 may be disposed along a top edge 160 of the discharge opening 116 (between the discharge opening 116 and the top side 134). The location of the tab 111 enables the tab 111 to hold open the incision 206 and at least partially guide the movement of the implant device 108 into the subdermal pocket 202 without requiring a bulky structure that forms an enclosed tunnel.

With reference now back to FIG. 6, the tab 111 may have a tapered shape along the length of the tab 111. For example, a thickness of the tab 111, which is a dimension along the height axis 191, tapers in a direction from the proximal end 156 to the distal end 140. The tapering optionally may be uniform along the length. Alternatively, the tapering may be non-uniform, such that some portions taper and other portions do not taper or at least taper at a different rate or slope.

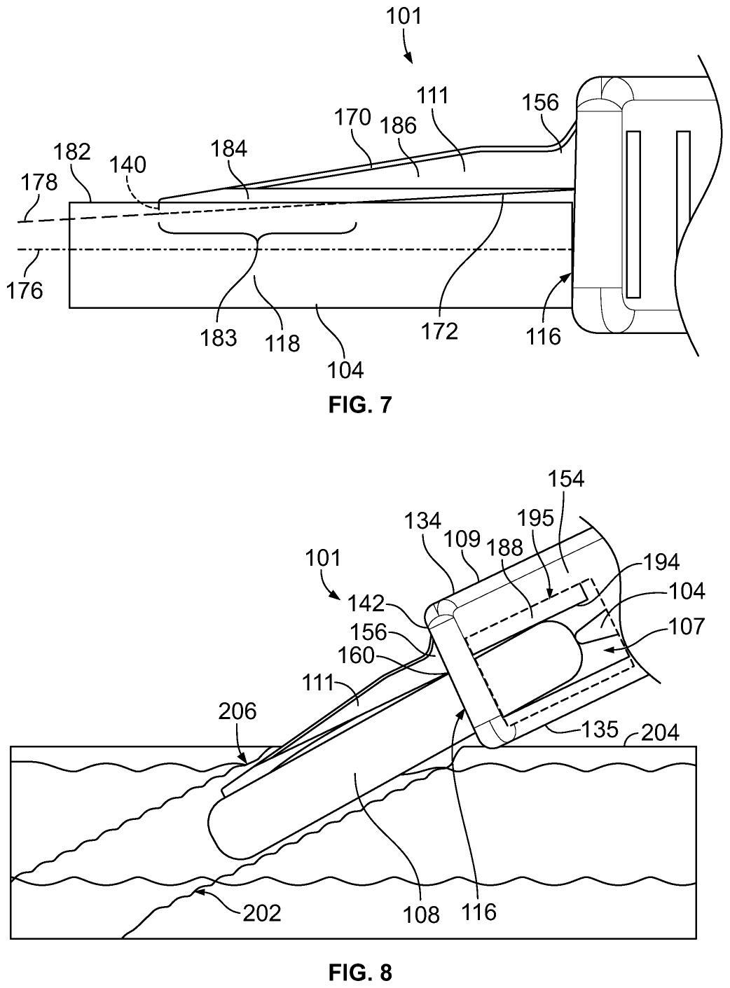

FIG. 7 is a side view of a portion of the implant delivery instrument 101 according to the embodiment shown in FIGS. 2 through 6. The side view shows that the thickness of the tab 111 gradually decreases from the proximal end 156 to the distal end 140. An upper surface 170 of the tab 111 faces away from the shaft 118 of the obturator 104. The upper surface may include one or more planar segments. The upper surface 170 provides a ramp that avoids catching or snagging tissue as the tab 111 is inserted into the incision.

The tab 111 has a lower surface 172 opposite the upper surface 170. In an embodiment, the lower surface 172 is angled and/or curved towards the obturator 104. For example, the distal end 140 of the tab 111 is closer to a center 176 (e.g., centerline) of the passage 107 (shown in FIG. 2) than the proximal end 156 of the tab 111. In FIG. 7, the lower surface 172 projects along a trajectory 178 that is transverse to the center 176 of the passage 107 (which is also the center of the discharge opening 116).

With additional reference to FIG. 6, the shaft 118 of the obturator 104 according to one or more embodiments defines an elongated groove 180 along an upper surface 182 of the shaft 118. The groove 180 is coaxial with the orientation of the shaft 118 and extends for at least a majority of the length of the shaft 118. In the illustrated embodiment, the groove 180 stops at the distal tip 126 of the shaft 118. The lower surface 172 of the tab 111 is configured to enter the groove 180. For example, as shown in FIG. 7, the distal end 140 of the tab 111 is received in the groove 180. A segment 183 of the lower surface 172 of the tab 111 within the groove 180 (e.g., below the upper surface 182 of the shaft 118) is depicted in phantom in FIG. 7. By the tab 111 nesting within the groove 180 of the shaft 118, the instrument 101 provides a smooth transition from the obturator 104 to the tab 111 when the instrument 101 is inserted into the patient. For example, the nesting enables the patient tissue at the incision to ride up the tab 111 from the shaft 118 without catching, snagging, or pinching at the interface.

In an embodiment, the tab 111 includes a narrow rib 184 that projects from a base 186 of the tab 111. The rib 184 may represent or define the segment 183 of the lower surface 172 that enters the groove 180 of the shaft 118. The base 186 is wider than the rib 184. The base 186 extends above the upper surface 182 of the shaft 118 without entering the groove 180.

As shown in FIG. 6, the main body section 109 of the housing 102 includes at least one device support wall 188 proximate to the front end 112. The device support wall(s) 188 define a ceiling 194 of the passage 107. The housing 102 has two device support walls 188 in the illustrated embodiment, but may have only one or more than two in alternative embodiments. The device support walls 188 are designed to support guiding the implant device 108 during the implantation procedure. The device support walls 188 are disposed proximate to the leading edge 142 of the main body section 109. For example, the device support walls 188 may be disposed between the receptacle cavity 128 and the leading edge 142.

With additional reference to FIG. 8, one device support wall 188 is shown in a cut-out area 195 of the gripping side of the housing 102. The cut-out area 195 is shown for descriptive purposes only, as the instrument 101 does not actually have a cut-out in the gripping side 154. The device support walls 188 provide a ceiling 194 of the passage 107. During the injection of the implant device 108, the ceiling 194 engages the implant device 108 and inhibits tilting of the implant device 108 in a direction away from the tab 111 as the obturator 104 pushes the implant device 108 through the discharge opening 116. For example, the tab 111 blocks the leading end of the implant device 108 from tilting upward relative to a direction of insertion, but cannot prevent the implant device 108 from tilting downward away from the tab 111. This latter function is provided by the device support walls 188. For example, the front of the implant device 108 cannot tilt downward because the back of the implant device 108 abuts against the ceiling 194.

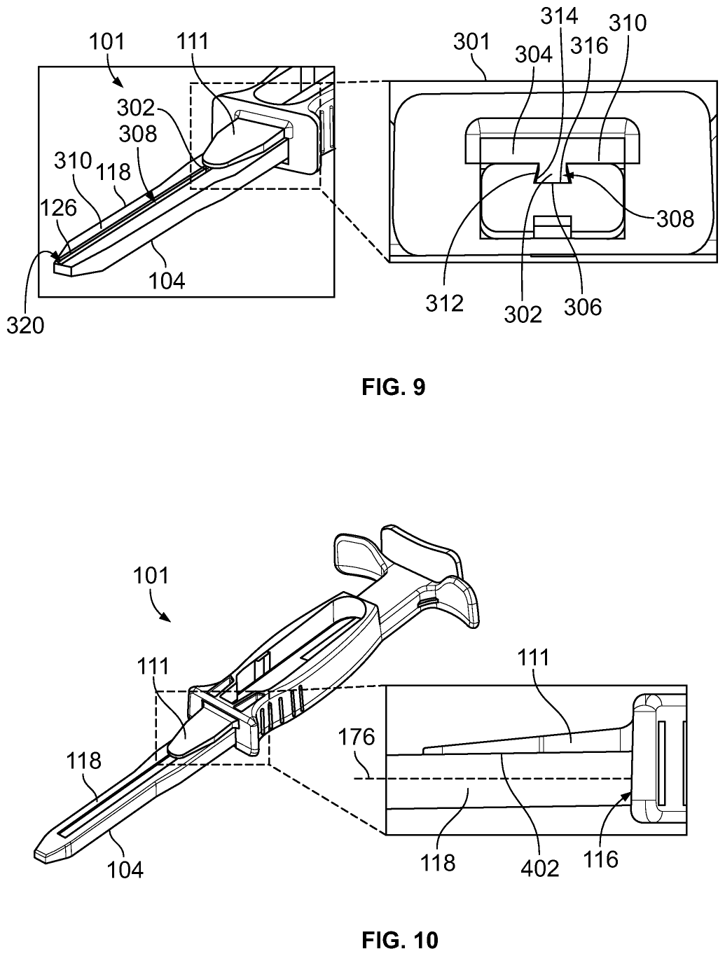

FIG. 9 is a perspective view of portion of the implant delivery instrument 101 according to a second embodiment. The instrument 101 in FIG. 9 is similar to the instrument 101 shown in FIGS. 2 through 8 except in tab 111 and the shaft 118 of the obturator 104. In the illustrated embodiment, the tab 111 has a narrow rib 302 that projects from a base 304 of the tab 111. The rib 302 defines a lower surface 306 of the tab 111. As shown in the enlarged inset portion 301 of FIG. 9, the narrow rib 302 is received within a groove 308 along an upper surface 310 of the shaft 118. The base 304 extends above the upper surface 310 of the shaft 118 without entering the groove 308.

Unlike the embodiment shown in FIGS. 2 through 8, the rib 302 and the groove 308 are interlocking to prevent separation of the tab 111 and the shaft 118. For example, the rib 302 has angled sides 312 that are complementary to respective angled walls 314 of the groove 308. Furthermore, the angled walls 314 are angled such that the groove 308 is wider at a bottom 316 of the groove 308 than at the upper surface 310. The rib 302 is wider at the lower surface 306 than at the connection to the base 304. The interlocking connection is a dovetail in the illustrated embodiment, but may have another shape in other embodiments. The rib 302 is configured to enter the groove 308 through an inlet lead-in section 320 at the distal tip 126 when the obturator 104 is pushed towards the advanced position.

FIG. 10 is a perspective view of the implant delivery instrument 101 according to a third embodiment. The third embodiment is similar to the instrument 101 shown in FIGS. 2 through 8 except in the tab 111. For example, an entirety of the tab 111 in FIG. 10 remains above the shaft 118 of the obturator 104. No portion of the tab 111 enters into a groove along the shaft 118. The tab 111 is not angled or curved towards the center 176 of the passage 107 (e.g., discharge opening 116), as shown in FIG. 7. Instead, the lower surface 402 of the tab 111 is linear and parallel to the center 176. The tab 111 may have a planar lower surface 402 that lacks any rib. The tab 111 is tapered to provide a smooth transition from the shaft 118 onto the tab 111 when the instrument 101 is inserted into the patient through the incision.

Optionally, the tabs 111 shown in FIGS. 9 and 10 may be wider than the tab 111 shown in FIGS. 2 through 8. Still, the tabs 111 in FIGS. 9 and 10 are no wider than the width of the discharge opening 116, at least along 90% of the length of each of the tabs 111.

A method may be performed to provide an implant delivery instrument. The method may include forming or obtaining a housing like the housing 102 shown and described with reference to FIGS. 2 through 10. For example, the housing 102 may be formed via additive manufacturing, molding, or the like. The housing 102 includes a tab 111 that is connected to a front end 112 of a main body section 109, as described with reference to FIGS. 2 through 10. An obturator 104 is loaded into the housing 102 to assemble the implant delivery instrument. An implant device 108 is loaded into a receptacle cavity of the housing 102 in preparation for implanting the device 108 into a subdermal pocket of a patient. The implant delivery instrument 101, as formed/obtained and assembled, can be used to inject the implant device 108 into the subdermal pocket with accuracy and reliability, and with less tissue damage and/or discomfort caused to the patient compared to known delivery tools.

In an alternative embodiment, the implant device may be a leadless implantable medical device that include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method Including The Same”, which are hereby incorporated by reference. Additionally or alternatively, the implant device may be a leadless cardiac monitor (ICM) that does not deliver electrical stimulation therapy (e.g., pacing or shock therapy). The ICM does not require leads to be implanted in the patient's heart. The ICM may include one or more structural and/or functional aspects of the device(s) described in U.S. patent application having Docket No. A15E1059, U.S. patent application Ser. No. 15/084,373, filed Mar. 29, 2016, entitled, “Method And System To Discriminate Rhythm Patterns In Cardiac Activity.”

All references, including publications, patent applications and patents, cited herein are hereby incorporated by reference to the same extent as if each reference were individually and specifically indicated to be incorporated by reference and were set forth in its entirety herein.

As will be appreciated by one skilled in the art, various aspects may be embodied as a system, method, or instrument/product. Aspects are described herein with reference to the figures, which illustrate example methods, devices, and systems according to various example embodiments. Accordingly, while various arrangements are broadly contemplated, described and illustrated herein, it should be understood that they are provided merely in illustrative and non-restrictive fashion, and furthermore can be regarded as but mere examples of possible working environments in which one or more arrangements may function or operate.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implant delivery instrument comprising:
a housing having a main body section that defines a passage therethrough from a front end of the main body section to a back end of the main body section, the passage configured to receive an obturator through an opening at the back end, the passage also configured to receive an implant device, wherein movement of the obturator through the passage is configured to push the implant device through a discharge opening at the front end and through an incision into a subdermal pocket of a patient,
the housing including a tab connected to and projecting from the front end of the main body section, wherein the tab has a blunt dissection tip for maintaining the incision in an open state without creating or enlarging the incision, and the tab is configured to surround the implant device moving through the discharge opening along only one side of the implant device.

2. The implant delivery instrument of claim 1, wherein a thickness of the tab tapers along a length of the tab from a proximal end of the tab connected to the main body section to a distal end of the tab.

3. The implant delivery instrument of claim 1, wherein a width of the tab, along at least 90% of a length of the hub, is no greater than a width of the discharge opening.

4. The implant delivery instrument of claim 1, wherein the front end of the main body section includes a leading edge that defines a hard stop surface, and the tab is connected to the leading edge.

5. The implant delivery instrument of claim 4, wherein the tab is rigid and integrally connected to the hard stop surface.

6. The implant delivery instrument of claim 1, wherein the main body section has a top side and a bottom side opposite the top side, the bottom side configured to face towards skin of the patient as the implant device is pushed through the discharge opening into the subdermal pocket, wherein the tab is disposed along a top edge of the discharge opening between the discharge opening and the top side of the main body section.

7. The implant delivery instrument of claim 6, wherein the main body section of the housing includes at least one device support wall proximate to the front end, the at least one device support wall defining a ceiling of the passage to engage the implant device and inhibit tilting of the implant device in a direction away from the tab as the obturator pushes the implant device through the discharge opening.

8. The implant delivery instrument of claim 1, wherein a lower surface of the tab is one of angled or curved so that a distal end of the tab is closer to a center of the passage than a proximal end of the tab connected to the main body section.

9. The implant delivery instrument of claim 8, further comprising the obturator, wherein the obturator includes a shaft that defines an elongated groove along an upper surface of the shaft, and the lower surface of the tab is configured to enter the groove.

10. The implant delivery instrument of claim 9, wherein the tab includes a narrow rib that projects from a base of the tab and defines the lower surface of the tab so that the narrow rib enters the groove of the shaft and the base of the tab extends above the upper surface of the shaft without entering the groove.

11. The implant delivery instrument of claim 10, wherein the narrow rib and the groove are interlocking to prevent separation of the tab and the shaft.

12. The implant delivery instrument of claim 1, wherein the main body section of the housing defines a receptacle cavity in communication with the passage, the receptacle cavity exposed to an external environment through a cavity opening along a side of the main body section, the receptacle cavity configured to receive the implant device through the cavity opening.

13. An implant delivery instrument comprising:
an obturator including an elongated shaft; and
a housing having a main body section that has a top side and a bottom side opposite the top side, the main body section defining a passage therethrough from a front end of the main body section to a back end of the main body section, the passage configured to receive the obturator through an opening at the back end, the passage also configured to receive an implant device, wherein movement of the obturator through the passage is configured to push the implant device through a discharge opening at the front end and through an incision into a subdermal pocket of the patient,
the housing including a tab connected to and projecting from the front end of the main body section, wherein the bottom side of the main body section is configured to face towards skin of the patient as the implant device is pushed into the subdermal pocket, and the tab connects to the front end at a location between the discharge opening and the top side of the main body section.

14. The implant delivery instrument of claim 13, wherein the tab has a blunt dissection tip for maintaining the incision in an open state without creating or enlarging the incision.

15. The implant delivery instrument of claim 13, wherein the tab is configured to surround the implant device moving through the discharge opening along only one side of the implant device.

16. The implant delivery instrument of claim 13, wherein a width of the tab, along at least 90% of a length of the hub, is no greater than a width of the discharge opening.

17. The implant delivery instrument of claim 13, wherein the main body section of the housing includes at least one device support wall proximate to the front end, the at least one device support wall defining a ceiling of the passage to engage the implant device and inhibit tilting of the implant device in a direction away from the tab as the obturator pushes the implant device through the discharge opening.

18. An implant delivery instrument comprising:
an obturator including a shaft that defines an elongated groove along a surface of the shaft; and
a housing having a main body section that defines a passage therethrough from a front end of the main body section to a back end of the main body section, the passage configured to receive the obturator through an opening at the back end, the passage also configured to receive an implant device, wherein movement of the obturator through the passage is configured to push the implant device through a discharge opening at the front end and through an incision into a subdermal pocket of a patient,
the housing including a tab connected to the front end of the main body section at a location between the discharge opening and a first side of the main body section, wherein the tab projects from the front end and is one of angled or curved so that a portion of the tab is received in the groove of the shaft of the obturator along a section of the shaft that projects out of the discharge opening.

19. The implant delivery device of claim 18, wherein the tab has a blunt dissection tip for maintaining the incision in an open state without creating or enlarging the incision, and the tab is configured to surround the implant device moving through the discharge opening along only one side of the implant device.

20. The implant delivery device of claim 18, wherein a thickness of the tab tapers along a length of the tab from a proximal end of the tab connected to the main body section to a distal end of the tab, and the distal end of the tab is received in the groove to provide a smooth transition from the obturator to the tab.
